(19) United States
(12) Patent Application Publication
(10) Pub. No.: US 2018/0289002 A1
(43) Pub. Date: Oct. 11, 2018

(54) METHOD OF PREPARING AMIDOAMINE ALKOXYLATES AND COMPOSITIONS THEREOF

(71) Applicant: Huntsman Petrochemical LLC, The Woodlands, TX (US)

(72) Inventors: Alan J. STERN, Magnolia, TX (US); Curtis M. ELSIK, The Woodlands, TX (US)

(21) Appl. No.: 16/009,702

(22) Filed: Jun. 15, 2018

Related U.S. Application Data
(63) Continuation of application No. 14/857,507, filed on Sep. 17, 2015.
Application No. 14/857,507 is a division of application No. 12/747,555, filed on Jun. 11, 2010, now abandoned.
(60) Provisional application No. 61/016,187, filed on Dec. 21, 2007.

(51) Int. Cl. A01N 25/30; A01N 57/20; C07C 233/36

(52) U.S. Cl. CPC A01N 25/30; A01N 57/20; C07C 233/36

(57) ABSTRACT

A method of producing an amidoamine alkoxylate is disclosed that involves reacting a triglyceride, a carboxylic acid, or a combination of triglyceride and carboxylic acid with an alkyleneamine to produce an amidoamine and alkoxylating the amidoamine with an alkylene oxide. Also disclosed are amidoamine alkoxylate compositions and their use in agricultural formulations.

### CROSS-REFERENCE TO RELATED APPLICATIONS

This application is a continuation of U.S. patent application Ser. No. 14/857,507, filed Sep. 17, 2015, which is a divisional of U.S. patent application Ser. No. 12/747,555, filed Jun. 11, 2010, which is the national stage entry of international Application Serial No. PCT/US2008/87574, filed Dec. 19, 2008, which claims the benefit of U.S. Provisional Patent Application Ser. No. 61/016,187, filed Dec. 21, 2007. The above-referenced patent applications are herein incorporated by reference in their entirety.

### BACKGROUND OF THE INVENTION

### Field of the Invention

The present invention is directed to an amidoamine alkoxylates, method of preparing such alkoxylates, and the use of such alkoxylates in agricultural applications.

### BACKGROUND OF THE INVENTION

Agricultural formulations typically include active ingredients. To these formulations adjuvants are typically added to enhance the effectiveness of the active ingredients. Adjuvants may be used as surfactants, extenders, wetting agents, sticking agents and fogging agents.

Current adjuvants have many drawbacks. For example, current alkoxylate adjuvants, like tallowamine alkoxylate with 15 ethylene oxide units, work well in low concentration (360 grams acid equivalent per liter (gae/L)) glyphosate-isopropylamine formulations, but are unable to be used effectively in high concentration glyphosate-potassium salts.

Thus, efforts are continually being made to define new and improved agricultural adjuvants and methods and processes of making them in order to improve cost, handling, compatibility, and/or other properties of such adjuvants.

### BRIEF SUMMARY OF SOME OF THE PREFERRED EMBODIMENTS

In one embodiment of the present invention, an amidoamine alkoxylate is disclosed having the formula:

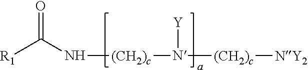

wherein a=1-3; c=2-3; R₁=C₅ to C₁₉ alkyl radical and wherein Y is each independently: H,

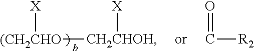

wherein X is each independently H, CH₃ or C₂H₅, b=0-10 and R₂=C₅ to C₁₉ alkyl radical.

In another embodiment of the present invention, an agricultural composition includes an adjuvant with the composition listed above and a pesticide.

In one embodiment of the present invention, a method of producing an amidoamine alkoxylate is disclosed that involves reacting a triglyceride, a carboxylic acid, or a combination of triglyceride and carboxylic acid with an alkyleneamine to produce an amidoamine and alkoxylating the amidoamine with an alkylene oxide.

In another embodiment of the present invention, a method of killing or controlling a pest is disclosed that contacts the agricultural composition listed above to the pest.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other compositions or methods for carrying out the same purposes of the present invention. It should also be realized by those skilled in the art that such equivalent compositions or methods do not depart from the spirit and scope of the invention as set forth in the appended claims.

### DETAILED DESCRIPTION OF THE PREFERRED EMBODIMENTS

In an embodiment of the present invention, an amidoamine alkoxylate composition is disclosed. The amidoamine alkoxylate has the formula:

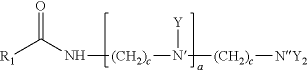

wherein a=1-3; c=2-3; R₁=C₅ to C₁₉ alkyl radical and wherein Y is each independently: H,

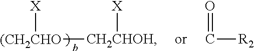

wherein X is each independently H, CH₃ or C₂H₅, b=0-10 and R₂=C₅ to C₁₉ alkyl radical.

In embodiments of the present invention, at least one of N′ and N″ is quaternized. In another embodiment of the present invention, at least one of N′ and N″ has been oxidized to form an amine oxide. One skilled in the art, with the benefit of this disclosure, will recognize other possible variations of the above composition.

Such an amidoamine alkoxylate may find beneficial use in numerous applications, including without limitation, agrichemicals, coatings, polymers, resins, fuels, lubes, process additives, and gas treating. One skilled in the art, with the benefit of this application would recognize a suitable application for this amidoamine alkoxylate.

The amidoamine alkoxylate above may be produced by the following method. The sequence below shows how the intermediate amidoamine is made from a triglyceride by reaction with an alkyleneamine.

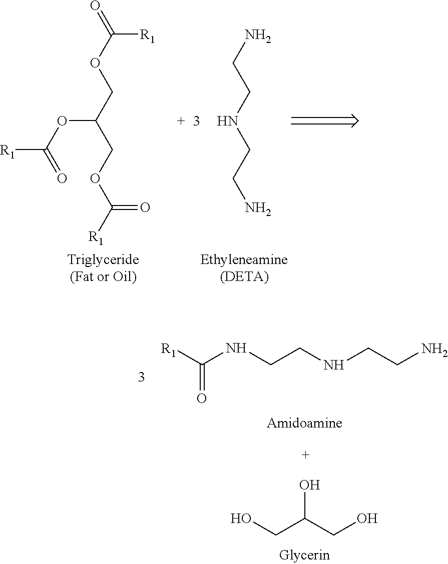

The triglyceride can be selected from oils or fats. Suitable triglycerides may be obtained from both plant and animal sources. Coconut oil, canola oil and palm oil may be used as the triglyceride. R₁ in the triglyceride above may be a fatty alkyl group. Furthermore, fatty acids could be used in place of fats and oils, with some modifications to the process. In another embodiment, a carboxylic acid, such as 2-ethylhexanoic acid may be used in place of the triglyceride. In another embodiment, a combination of triglyceride and carboxylic acid may be used to react with the alkyleneamine. One skilled in the art, with the benefit of this disclosure, would recognize appropriate triglycerides, carboxylic acids and combinations thereof for use in embodiments of the present invention.

The alkyleneamine may be selected from various amines, including ethyleneamine. Such ethyleneamines may include diethylenetriamine (DETA), tetraethylenetriamine (TETA), and tetraethylene pentamine (TEPA). DETA is shown in the sequence above. Dipropylene triamine may also be used. One skilled in the art, with the benefit of this disclosure, would recognize appropriate alkyleneamines for use in embodiments of the present invention.

For the above reaction, it is recommended to have at a minimum the molar ratio of triglyceride to alkyleneamine of 1:3, however, it is preferable to use an excess of the alkyleneamine component.

The intermediate product mixture contains the amidoamine plus glycerin, and other minor products (not shown). It is recognized that some diamide will also form during the reaction. Diamide formation may be desirable when the alkyleneamine is TETA or TEPA. The amount of diamide may vary depending on the ratios of triglyceride to alklyeneamine used.

The amidoamine is then alkoxylated with an alkylene oxide, such as ethylene oxide, as shown below. In another embodiment, the alkylene oxide may include propylene oxide. One skilled in the art, with the benefit of this disclosure, would recognize other appropriate alkylene oxides for use in embodiments of the present invention.

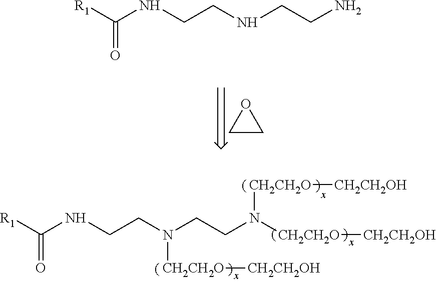

In this structure, x can be from 0 to 5, preferably 0 to 2

Once formed, the amidoamine alkoxylate may be used alone or combine with other chemicals for use in a variety of applications. The amidoamine alkoxylate may be used as an adjuvant in agriculture applications.

In one embodiment of the present invention, an agricultural composition is disclosed that includes a pesticide and an amidoamine alkoxylate of the formula:

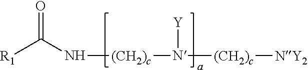

wherein a=1-3; c=2-3; R₁=C₅ to C₁₉ alkyl radical and wherein Y is each independently: H,

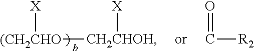

wherein X is each independently H, CH₃ or C₂H₅, b=0-10 and R₂=C₅ to C₁₉ alkyl radical.

The pesticide encompasses all agriculturally active ingredients and combinations of such active ingredients. In an embodiment of the present invention the pesticide is a herbicide.

In one embodiment of the present invention, the pesticide is glyphosate. In another embodiment of the present invention, the pesticide is glufosinate and salts thereof. In another embodiment, the pesticide is a combination of glyphosate and glufosinate. Any agriculturally acceptable glyphosate (or glufosinate) salt can be used, but the most preferred salts are isopropylamine salts, potassium salts, and ammonium salts. Generally, a high concentration of glyphosate salt (or glufosinate salt) is desired, as long as the resulting blend is homogenous, has a suitable viscosity, and is physically stable over the temperature range −20° C. to 60° C. High concentrations of glyphosate may be considered about 450 grams acid equivalent per liter (gae/L) ae and above. Another high concentration that is typically used is about 540 gae/L. A high glyphosate concentration example can be made from 58% potassium glyphosate solution (84 parts), water (8 parts), and adjuvant (8 parts). This formulation contains 540 gae/L glyphosate acid. More or less of each component will also work.

Useful ranges for the amidoamine alkoxylate in glyphosate salt aqueous formulations may be from about 1% to about 25% on a weight basis. However, the amount of amidoamine alkoxylates included, for example, in the weed killing formulation, will depend on several factors such as the amount of glyphosate present.

To the agricultural composition described above other active ingredients, additives and solvents may be added. One skilled in the art would recognize appropriate active ingredients, additives and solvents that may be combined with this agricultural composition.

Also disclosed is a method of killing or controlling pests. The agricultural composition listed above is contacted with the pest. The agricultural composition may be used in the above listed for or diluted with water or an appropriate diluent.

The following specific examples illustrating the best currently-known method of practicing this invention are described in detail in order to facilitate a clear understanding of the invention. It should be understood, however, that the detailed expositions of the application of the invention, while indicating preferred embodiments, are given by way of illustration only and are not to be construed as limiting the invention since various changes and modifications within the spirit of the invention will become apparent to those skilled in the art from this detailed description.

### EXAMPLES

### Example 1

Synthesis of amidoamine alkoxylate. In a 2 liter, 3 neck round bottom flask equipped with a nitrogen inlet and overhead mechanical agitation was placed 952 grams of molten coconut oil (Columbus Foods Company of Chicago, Ill.) and 448 grams of diethylenetriamine (Huntsman Corporation). Under a nitrogen blanket, this was stirred and heated for 4 hours at 150° C. The intermediate product (called the amidoamine) solidified to an oily yellow paste upon cooling. A 976 gram portion of this intermediate product was transferred to a reactor for ethoxylation. Under a nitrogen blanket, the amidoamine was heated to 150° C. and the reactor charged with 803 grams of ethylene oxide (EO) over a one hour period. During the EO addition, the reaction temperature was allowed to rise to 160° C. When the ethylene oxide addition was complete, the reactor was maintained at 150° C. for an additional 2.5 hours to digest any remaining EO. It was then cooled to 115° C. and discharged from the reactor, yielding a thick, clear amber liquid.

### Example 2

Synthesis of amidoamine alkoxylate. In a 2 liter, 3 neck round bottom flask equipped with a nitrogen inlet and overhead mechanical agitation was placed 896 grams of molten coconut oil (Columbus Foods Company of Chicago, Ill.) and 504 grams of diethylenetriamine (Huntsman Corporation). Under a nitrogen blanket, this was stirred and heated for 4 hours at 150° C. The intermediate product (called the amidoamine) solidified to an oily yellow paste upon cooling. A 976 gram portion of this intermediate product was transferred to a reactor for ethoxylation. Under a nitrogen blanket, the amidoamine was heated to 150° C. and the reactor charged with 944 grams of EO over a one hour period. During the EO addition, the reaction temperature was allowed to rise to 160° C. When the EO addition was complete, the reactor was maintained at 150° C. for an additional 2.5 hours to digest any remaining EO. It was then cooled to 115° C. and discharged from the reactor, yielding a thick, clear amber liquid.

### Example 3

Synthesis of amidoamine alkoxylate. In a 2 liter, 3 neck round bottom flask equipped with a nitrogen inlet and overhead mechanical agitation was placed 840 grams of molten coconut oil (Columbus Foods Company of Chicago, Ill.) and 560 grams of diethylenetriamine (Huntsman Corporation). Under a nitrogen blanket, this was stirred and heated for 4 hours at 150° C. The intermediate product (called the amidoamine) solidified to an oily yellow paste upon cooling. A 976 gram portion of this intermediate product was transferred to a reactor for ethoxylation. Under a nitrogen blanket, the amidoamine was heated to 150° C. and the reactor charged with 1076 grams of EO over a one hour period. During the EO addition, the reaction temperature was allowed to rise to 160° C. When the ethylene oxide addition was complete, the reactor was maintained at 150° C. for an additional 2.5 hours to digest any remaining EO. It was then cooled to 115° C. and discharged from the reactor, yielding a thick, clear amber liquid.

For the above reactions, appropriate ranges for the alkyleneamine (A), the triglyceride (B), and alkylene oxide (C) can be expressed in terms of weight ratios. When A is coconut oil and B is DETA, the preferred ratio of A to B is from 3:1 to 1:1. In examples 1-3, the ratios of A:B:C are as follows in Table 1.

TABLE 1
Ratios of alkyleneamine, triglyceride and alkylene oxide

| | | B (Coconut | C (Ethylene |
|---|---|---|---|
| Example | A (DETA) | oil) | oxide) |
| 1 | 18 | 37 | 45 |
| 2 | 18 | 33 | 49 |
| 3 | 19 | 29 | 52 |

Examples 4-10 show agricultural formulations of amidoamine alkoxylate and glyphosate salts. The percentages refer to weight percent.

### Example 4

| Component | Percent |
|---|---|
| Amidoamine Alkoxylate from Example 1 | 8 |
| Water | 8 |
| 58% potassium glyphosate liquid | 84 |

Result: a clear, homogenous, fluid yellow liquid; 540 gae/L.

### Example 5

| Component | Percent |
|---|---|
| Amidoamine Alkoxylate from Example 1 | 10 |
| Water | 23.9 |
| 62% glyphosate-isopropylamine salt liquid | 66.1 |

Result: a clear, homogenous, fluid yellow liquid; 360 gae/L.

### Example 6

| Component | Percent |
|---|---|
| Amidoamine Alkoxylate from Example 2 | 8 |
| Water | 8 |
| 58% potassium glyphosate liquid | 84 |

Result: a clear, homogenous, fluid yellow liquid; 540 gae/L.

### Example 7

| Component | Percent |
|---|---|
| Amidoamine Alkoxylate from Example 2 | 10 |
| Water | 23.9 |
| 62% glyphosate-isopropylamine salt liquid | 66.1 |

Result: a clear, homogenous, fluid yellow liquid; 360 gae/L.

### Example 8

| Component | Percent |
|---|---|
| Amidoamine Alkoxylate from Example 3 | 8 |
| Water | 8 |
| 58% potassium glyphosate liquid | 84 |

Result: a clear, homogenous, fluid yellow liquid; 540 gae/L.

### Example 9

| Component | Percent |
|---|---|
| Amidoamine Alkoxylate from Example 3 | 10 |
| Water | 23.9 |
| 62% glyphosate-isopropylamine salt liquid | 66.1 |

Result: a clear, homogenous, fluid yellow liquid; 360 gae/L.

### Example 10

| Component | Percent |
|---|---|
| Amidoamine Alkoxylate from Example 2 | 10 |
| Water | 0 |
| 58% potassium glyphosate liquid | 90 |

Result: a clear, homogenous, fluid yellow liquid, density = 1.407 g/ml at 22 C.; 600 gae/L.

Examples 4, 6, 8 and 10 show the amidoamine alkoxylate is surprisingly compatible with highly concentrated potassium glyphosate solutions. The most concentrated known liquid potassium glyphosate formulation in the prior art is 540 gae/L of glyphosate. Using the new adjuvant, 600 gae/L is easily achieved.

Examples 5, 7, and 9 show the amidoamine alkoxylate is compatible with highly concentrated isopropylamine glyphosate solutions.

Surprisingly, Examples 4, 6, 8 and 10 show that with the new amidoamine alkoxylate it is possible to make potassium glyphosate liquid formulations that are fluid (low viscosity) and highly concentrated.

The new amidoamine alkoxylate are also very electrolyte tolerant, if in their protonated (cationic) form. Thus, it is possible to make a blend of ammonium sulfate, acetic acid, and the surfactant in water at high concentration. Example 11 shows such a blend. Other acids will work in place of acetic acid. The percentages refer to weight percent.

### Example 11

| Component | Percent |
|---|---|
| Ammonium sulfate | 20 |
| Glacial acetic acid | 5 |
| Amidoamine Alkoxylate from Example 1 | 25 |
| Water | 50 |

Result: a clear, homogenous, fluid yellow liquid.

### Bioefficacy of the Amidoamine Alkoxylate With Glyphosate

A formulation of glyphosate and the amidoamine alkoxylate was successfully tested in field trials and showed positive results. The plant species in the test were: tall waterhemp, velvetleaf, ivyleaf, morning glory, common cocklebur, and dent corn.

### Example 12

Synthesis of amidoamine alkoxylate. In a 2 liter, 3 neck round bottom flask equipped with a nitrogen inlet and overhead mechanical agitation was placed canola oil, 1000 grams, and diethylenetriamine (Huntsman Corporation), 500 grams. Under a nitrogen blanket, this was stirred and heated for 90 minutes at 160° C. The reaction mixture (called the amidoamine) was cooled to 80° C. The amidoamine was transferred to a reactor for ethoxylation. After purging the reactor with nitrogen gas, the amidoamine was heated to 140° C. and the reactor charged with 1.5 kg of ethylene oxide over a 90 minute period. During the EO addition, the reaction temperature was allowed to rise to 147° C. When the ethylene oxide addition was complete, the reactor was maintained at 145° C. for an additional 2.0 hours to digest any remaining EO. It was then cooled to 115° C. and discharged from the reactor, yielding a thick, clear amber liquid.

### Example 13

Synthesis of amidoamine alkoxylate from a carboxylic acid. In a 2 liter, 3 neck round bottom flask equipped with a nitrogen inlet and overhead mechanical agitation and a Dean-Stark water collector was placed 2-ethylhexanoic acid, 672 grams, (Sigma-Aldrich, Inc. of St. Louis, Mo.) and diethylenetriamine (Huntsman Corporation), 600 grams. This was stirred and heated for 2 hours at 180° C. and then for 2 hours at 185° C. During this time, 93 grams of clear liquid was collected in the Dean-Stark apparatus. The reaction mixture (called the amidoamine) was cooled to 80° C. and 1.1 kg was transferred to a reactor for ethoxylation. After purging the reactor with nitrogen gas, the amidoamine was heated to 147° C. and the reactor charged with 0.9 kg of ethylene oxide over a 90 minute period. When the ethylene oxide addition was complete, the reactor was maintained at 147° C. for an additional 2.0 hours to digest any remaining EO. It was then cooled to 115° C. and discharged from the reactor, yielding a thick, clear amber liquid.

### Example 14

Synthesis of amidoamine alkoxylate. In a 2 liter, 3 neck round bottom flask equipped with a nitrogen inlet and overhead mechanical agitation was placed coconut oil 603.5 grams (Columbus Foods Company of Chicago, Ill.) and dipropylene triamine, 510 grams (Sigma-Aldrich, Inc. of St. Louis, Mo.). Under a gentle nitrogen stream, this was stirred and heated for 4.5 hours at 160° C. The reaction mixture (called the amidoamine) was cooled to 80° C. and 1.0 kg was transferred to a reactor for ethoxylation. After purging the reactor with nitrogen gas, the amidoamine was heated to 160° C. and the reactor charged with 1.0 kg of ethylene oxide over a 90 minute period. When the ethylene oxide addition was complete, the reactor was maintained at 160° C. for an additional 2.0 hours to digest any remaining EO. It was then cooled to 115° C. and discharged from the reactor, yielding a thick, clear amber liquid.

Examples 15-23 show agricultural formulations of amidoamine alkoxylate and glyphosate salts. The percentages refer to weight percent.

### Example 15

| Component | Percent |
|---|---|
| Adjuvant from example 12 | 8 |
| Water | 7 |
| 58% potassium glyphosate liquid | 85 |

Result: a clear, homogenous, fluid yellow liquid at 25° C. At 50° C., this blend separated into two phases.

### Example 16

| Component | Percent |
|---|---|
| Adjuvant from example 12 | 10 |
| Water | 23.9 |
| 62% glyphosate-isopropylamine salt liquid | 66.1 |

Result: a clear, homogenous, fluid amber-colored liquid at 21° C.

### Example 17

| Component | Percent |
|---|---|
| Adjuvant from example 13 | 8 |
| Water | 7 |
| 58% potassium glyphosate liquid | 85 |

Result: a clear, homogenous, fluid yellow liquid at 21° C.

### Example 18

| Component | Percent |
|---|---|
| Adjuvant from example 13 | 10 |
| Water | 23.9 |
| 62% glyphosate-isopropylamine salt liquid | 66.1 |

Result: a cloudy, inhomogeneous mixture at 21° C.

### Example 19

| Component | Percent |
|---|---|
| Adjuvant from example 13 | 25 |
| Glufosinate solution, 50% active | 36.1 |
| Propylene glycol methyl ether | 10 |
| Acetic acid, glacial | 7.2 |
| Water | 21.7 |

Result: a clear, homogenous, fluid yellow liquid at 21° C.

### Example 20

| Component | Percent |
|---|---|
| Adjuvant from example 14 | 8 |
| Water | 7 |
| 58% potassium glyphosate liquid | 85 |

Result: a clear, homogenous, fluid yellow liquid at 25° C.

### Example 21

| Component | Percent |
|---|---|
| Adjuvant from example 14 | 10 |
| Water | 23.9 |
| 62% glyphosate-isopropylamine salt liquid | 66.1 |

Result: a clear, homogenous, fluid amber-colored liquid at 25° C.

### Example 22

| Component | Percent |
|---|---|
| Adjuvant from example 14 | 25 |
| Glufosinate solution, 50% active | 36.1 |
| Propylene glycol methyl ether | 10 |
| Acetic acid, glacial | 7.2 |
| Water | 21.7 |

Result: a clear, homogenous, fluid yellow liquid at 25° C.

### Example 23

| Component | Percent |
|---|---|
| Adjuvant from example 13 | 25 |
| Glufosinate solution, 50% active | 65 |
| Acetic acid, glacial | 10 |

Result: a clear, homogenous, viscous yellow liquid at 21° C.

### Example 24

Oxidation of an amidoamine alkoxylate with hydrogen peroxide. The amidoamine alkoxylate of Example 12 (130 grams) was placed in a flask equipped with a mechanical stirrer. With continuous stirring, Versene 100 chelating agent (1 gram), (The Dow Chemical Company, Midland, Mich.) was added. Next, hydrogen peroxide (42 grams of a 35% peroxide solution in water) was added in small portions over a 120 minute period, taking care to keep the temperature of the reaction between 35° C. and 60° C. After the peroxide addition was complete, the mixture was stirred for an additional 120 minutes. The product is a viscous yellow liquid.

Examples 25-27 show pesticide formulations using the oxidized amidoamine alkoxylate. The percentages refer to weight percent.

### Example 25

| Component | Percent |
|---|---|
| Adjuvant from Example 24 | 10 |
| Water | 5 |
| 58% potassium glyphosate liquid | 85 |

Result: a clear, homogenous, slightly viscous, light yellow liquid

### Example 26

| Component | Percent |
|---|---|
| Adjuvant from Example 24 | 12 |
| Water | 21.9 |
| 62% glyphosate-isopropylamine salt liquid | 66.1 |

Result: a clear, homogenous, fluid, light yellow liquid at 21° C.

### Example 27

| Component | Percent |
|---|---|
| Adjuvant from Example 24 | 25 |
| Glufosinate ammonium solution, 50% active | 65 |
| Acetic acid, glacial | 10 |

Result: a clear, homogenous, slightly viscous yellow liquid at 21° C.

### Example 28

Viscosity of glyphosate salt formulations. The following Table 2 shows viscosity data for some of the example formulations. The data was obtained with a Brookfield DV-II viscometer, equipped with an LV-2 spindle, at 21° C., at 60 rpm. In the table below, DPT is used as an abbreviation for 3-aminopropyl-1,3-propanediamine. Measurements are in centipoise.

TABLE 2
Viscosity Measurements for selected Examples

| | Glyphosate Salt | | |
|---|---|---|---|
| Example | Type | Amidoamine type | cP |
| 6 | Potassium | Coconut/DETA | 62 |
| 7 | Isopropylamine | Coconut/DETA | 44 |
| 10 | Potassium | Coconut/DETA | 251 |
| 15 | Potassium | Canola/DETA | 167 |
| 16 | Isopropylamine | Canola/DETA | 66 |
| 17 | Potassium | 2- | 70 |
| | | ethylhexanoic/DETA | |
| 18 | Isopropylamine | 2- | 43 |
| | | ethylhexanoic/DETA | |
| 20 | Potassium | Coconut/DPT | 66.5 |
| 21 | Isopropylamine | Coconut/DPT | 43 |

Although the present invention and its advantages have been described in detail, it should be understood that various changes, substitutions and alterations may be made herein without departing from the spirit and scope of the invention as defined by the appended claims.

What is claimed is:

1. A method of producing a compound comprising the steps of:
reacting two or more reactants to produce an intermediate product; and
reacting the intermediate product with a third reactant to form the compound having the formula: 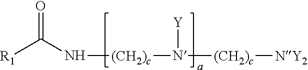
wherein a=1-3; c=2-3; R₁=C₅ to C₁₉ alkyl radical and wherein Y is each independently: 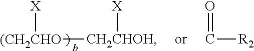
wherein X is each independently H, CH₃ or C₂H₅, b=0.37-10 and R₂=C₅ to C₁₉ alkyl radical.

2. The method of claim 1, wherein b=0.37-5.

3. The method of claim 1, wherein b=0.37-2.

4. A method of producing an amidoamine alkoxylate comprising the steps of:
reacting one or more reactants with an alkyleneamine to produce an amidoamine; and
alkoxylating the amidoamine to form the amidoamine alkoxylate having the formula: 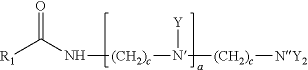
wherein a=1-3; c=2-3; R₁=C₅ to C₁₉ alkyl radical and wherein Y is each independently: 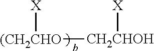
wherein X is each independently H, CH₃ or C₂H₅, b=0.37-10 and R₂=C₅ to C₁₉ alkyl radical.

5. The method of claim 4, wherein b=0.37-5.

6. The method of claim 4, wherein b=0.37-2.

7. The method of claim 4, wherein the alkyleneamine is ethyleneamine.

8. The method of claim 7, wherein the ethyleneamine is selected from the group consisting of: diethylenetriamine, tetraethylenetriamine, and tetraethylene pentamine.

9. The method of claim 4, wherein the amidoamine is alkoxylated with an alkylene oxide.

10. The method of claim 9, wherein the alkylene oxide comprises ethylene oxide.

11. A method of producing an amidoamine alkoxylate comprising the steps of:
reacting a triglyceride, a carboxylic acid, or a combination of triglyceride and carboxylic acid with an alkyleneamine to produce an amidoamine; and
alkoxylating the amidoamine with an alkylene oxide to form the amidoamine alkoxylate having the formula: 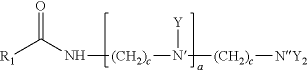
wherein a=1-3; c=2-3; R₁=C₅ to C₁₉ alkyl radical and wherein Y is each independently: 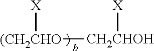
wherein X is each independently H, CH₃ or C₂H₅, b=0.37-10 and R₂=C₅ to C₁₉ alkyl radical.

12. The method of claim 11, wherein the triglyceride comprises a fat or an oil.

13. The method of claim 11, wherein the alkyleneamine is ethyleneamine.

14. The method of claim 13, wherein the ethyleneamine is selected from the group consisting of: diethylenetriamine, tetraethylenetriamine, and tetraethylenepentamine.

15. The method of claim 11, wherein the molar ratio of triglyceride to alkyleneamine is about 1:3.

16. The method of claim 11, wherein the molar ratio of triglyceride to alkyleneamine is greater than 1:3 favoring the alkyleneamine.

17. The method of claim 11, wherein the alkylene oxide comprises ethylene oxide.

18. The method of claim 17, wherein b=0.37-5.

19. The method of claim 11, wherein b=0.37-5.

20. The method of claim 11, wherein b=0.37-2.
